(19) United States
(12) Patent Application Publication
(10) Pub. No.: US 2021/0244651 A1
(43) Pub. Date: Aug. 12, 2021

(54) ANTI-WRINKLE COSMETIC COMPOSITION

(71) Applicant: NAL PHARMACEUTICAL GROUP LIMITED, Hong Kong SAR (HK)

(72) Inventor: Chun Kwong CHU, Hong Kong (CN)

(73) Assignee: NAL PHARMACEUTICAL GROUP LIMITED, Hong Kong SAR (HK)

(21) Appl. No.: 16/972,841

(22) PCT Filed: Jun. 5, 2019
(86) PCT No.: PCT/IB2019/054694

§ 371 Date: Dec. 7, 2020

Related U.S. Application Data
(60) Provisional application No. 62/681,143, filed on Jun. 6, 2018.

(51) Int. Cl. A61K 8/9789; A61Q 19/08; A61K 8/06; A61K 8/04

(52) U.S. Cl. CPC A61K 8/9789; A61Q 19/08; A61K 8/064; A61K 2800/49; A61K 8/042; A61K 2800/524; A61K 2800/522; A61K 8/062

(57) ABSTRACT

An anti-wrinkle cosmetic composition comprises a Kava extract, one or more penetration and other anti-wrinkle agents. The Kava extract is extracted from Piper Methysticum root, leaf, stem, or their combinations.

### CROSS-REFERENCE TO RELATED APPLICATION

This application claims the benefit of priority from U.S. Provisional Application Ser. No. 62/681,143, filed Jun. 6, 2018, the contents of which are incorporated herein by reference.

### FIELD OF INVENTION

The present invention relates to topical composition on skin that reduces the appearance of wrinkles and lines.

### BACKGROUND OF THE INVENTION

Wrinkles and lines are signs of aging that affect appearance and people are trying what they can do to minimize their formation. There are two different types of wrinkles: the static wrinkles and dynamic wrinkles. Static wrinkles are caused by the breakdown of collagen and elastic fibers due to aging and environmental factors such as sun exposure. Deep wrinkles are also associated with the build-up of the musculature below the skin surface. Moreover, repeating the facial muscle contraction can thicken the muscle layer and thus deepen the wrinkles. Dynamic wrinkles appear around the facial expression muscles when having facial expressions, that is, when these muscles contract. Skin becomes smooth again when these muscles relax. Examples of dynamic wrinkles are forehead, crow's feet, frown lines, chin dimpling, upper lip creases and down turned mouth corners. However, it is believed that there is still a slight contractile posture when the muscles are at rest because of the base-level or spontaneous quantal release of acetylcholine.

Kava is a root extract of Piper Methysticum that is used as a psychoactive drink in the South Pacific. The active substance in Kava is known as kavalactones, which have muscle relaxant, local anesthetic, anxiolytic and anticonvulsive properties. The action mechanism of Kava acting as a muscle relaxant is not fully understood. Yet studies indicated that Kava can disrupt neurotransmitter system by directly inhibiting voltage dependent ion channels and thus causing a release of muscle tension. Kavalactones can also interact with most neurotransmitter system, for instances, serotonergic and glutamatergic systems.

### SUMMARY OF THE INVENTION

The present invention relates to topical composition on skin that reduces the appearance of wrinkles and lines. The present invention comprises one or more plant extracts having muscle relaxant activity. The present invention further comprises one or more penetration enhancers for promoting permeation of active ingredients through skin.

### BRIEF DESCRIPTION OF THE DRAWINGS

FIG. 1 shows the fast acting anti-wrinkle effect of the invention.

FIG. 2 shows the long term anti-wrinkle effect of the invention.

FIG. 3 is a graph showing the wrinkle score change after applying the invention for 3 months.

FIG. 4A is a graph showing the percentage of subjects whose wrinkle was reduced after the application of the invention assessed by investigators. FIG. 4B is a graph showing the percentage of subjects whose wrinkle was reduced remarkably after the application of the invention by self-assessment.

FIG. 5 is a graph showing the percentage of the water content of skin around the eyes.

FIG. 6 is a graph showing the percentage of the elasticity of skin around the eyes.

### DETAILED DESCRIPTION OF THE INVENTION

The detailed description is merely exemplary in nature and is not intended to limit application and uses, The following examples further illustrate the present invention without, however, limiting the scope of the invention thereto. Various changes and modifications can be made by those skilled in the art on the basis of the description of the invention, and such changes and modifications are also included in the present invention.

One embodiment of the present invention relates to a cosmetic composition comprising a Kava extract to reduce the appearance of wrinkles and lines in a person's skin. The Kava extract is obtained from Piper Methysticum root, leaf, stem, or their combinations thereof. The Kava extract is extracted by using polar solvents, non-polar solvents, or supercritical fluid such as carbon dioxide. In particular embodiment of the invention, Kavalactones are the active substances. The amount of Kavalactones in the compositions of the present invention is about 1% to about 90% by weight of the Kava extract.

In certain embodiment of the invention further comprises other anti-wrinkle agents such as dipalmitoyl hydroxyproline, sodium hyaluronate, palmitoyl pentapeptide-3, acetyl hexapeptide-3, acetyl hexapeptide-8, and CoQ 10.

The cosmetic composition of the invention further comprises one or more penetration enhancers. Particularly, the penetration enhancers have a combined HLB value of about 1 to about 16. The penetration enhancer are such as PEG-8 beeswax, PEG-75 stearate, pegoxol-7 stearate, propylene glycol monocaprylate, propylene glycol monolaurate, propylene glycol monostearate, propylene glycol dioleate, 2-hydroxypropyl stearate, 2-hydroxypropyl laurate, propylene glycol oleate, propylene glycol distearate, propylene glycol dicaprylate, propylene glycol dilaurate, polypropylene glycol (17) dioleate, propyleneglycol monolaurate, propylene glycol monomyristate, dipropylene glycol dipelargonate, polypropylene glycol monobutyl ether oleate, propylene glycol dipelargonate, propylene glycol didecanoate, dipropylene glycol dipelargonate, propylene glycol bis(9,10-epoxystearate), propylene glycol monoisostearate, propylene glycol diundecanoate, glycol monoethyl ether, diethylene glycol monobutyl ether, oleoyl macrogolglycerides, lauroyl macrogolycerides, stearoyl macrogolgylycerides, caprylocaproyl macrogolglycerides, medium-chain triglycerides, polyglyceryl-3 diisostearate, polyglyceryl oleate, ethylene glycol paimitostearate, dissopropyl adipate, di-nbutyl adipate, dimethyl adipate, dimethyl isosorbide, or diethylene glycol monoethyl ether. The amount of the penetration enhancers is about 0.1% to about 20%, about 0.1% to about 15%, or about 1% to about 10% of the total weight of the composition.

Further, the cosmetic composition of the invention further comprises a surfactant, a humectant, an emulsifier, a solubilizing agent, a solvent, a base polymer, a diluent, an antioxidant, a fragrance, a secondary penetration enhancer or a preservative. The secondary penetration enhancer is selected from the group consisting of glycerol and terpenes.

The cosmetic composition of the present invention is in the form of a product selected from the group comprising O/W and W/O emulsions, lotions, ointments, paste, solutions, lacquers, creams or gels.

In another embodiment of the present invention relates to a method of reducing the appearance of wrinkles and lines in a person's skin. The method comprises topically applying the cosmetic composition comprising Kava extract to skin in need thereof, wherein at least a fraction of 1% to 80% or greater of the Kavalactones is delivered to and absorbed by the skin and absorbed systemically.

### Enhancer

The present invention comprises one or more penetration enhancers. The penetration enhancer is selected from group consisting of PEG-8 beeswax, PEG-75 stearate, pegoxol-7 stearate, propylene glycol monocaprylate, propylene glycol monolaurate, propylene glycol monostearate, propylene glycol dioleate, 2-hydroxypropyl stearate, 2-hydroxypropyl laurate, propylene glycol oleate, propylene glycol distearate, propylene glycol dicaprylate, propylene glycol dilaurate, polypropylene glycol (17) dioleate, propyleneglycol monolaurate, propylene glycol monomyristate, dipropylene glycol dipelargonate, polypropylene glycol monobutyl ether oleate, propylene glycol dipelargonate, propylene glycol didecanoate, dipropylene glycol dipelargonate, propylene glycol bis(9,10-epoxystearate), propylene glycol monoisostearate, propylene glycol diundecanoate, glycol monoethyl ether, diethylene glycol monobutyl ether, oleoyl macrogolglycerides, lauroyl macrogolycerides, stearoyl macrogolgylycerides, caprylocaproyl macrogolglycerides, medium-chain triglycerides, polyglyceryl-3 diisostearate, polyglyceryl oleate, ethylene glycol paimitostearate, dissopropyl adipate, di-nbutyl adipate, dimethyl adipate, dimethyl isosorbide, or diethylene glycol monoethyl ether. The amount of the penetration enhancers is about 0.1% to about 20%, about 0.1% to about 15%, or about 1% to about 10% of the total weight of the composition.

The cosmetic composition further comprises a secondary penetration enhancer such as glycerol and terpenes. The amount of the secondary penetration enhancer is about 0.1% to about 20%, about 0.1% to about 15%, or about 1% to about 10% of the total weight of the composition.

### Surfactant

The cosmetic composition of the invention further comprises the surfactant. The surfactant is such as anionic, catatonic, nonionic, amphoteric, saturated and unsaturated higher aliphatic acid salts (e.g., sodium laurate, sodium stearate, sodium oleate, sodium linolenate, etc), long-chain alkyl sulfate salts, alkylbenzenesulfonic acids (e.g., hexylbenzenesulfonic acid, octylbenzenesulfonic acid, dodecylbenzenesulfonic acid, etc) and their salts, polyoxyalkylene alkyl ether sulfate salts, polyoxyalkylene alkenyl ether sulfate salts, the salts of polyoxyethylene alkyl sulfate esters, the salts of the alkyl esters of sulfosuccinic acid, polyoxyalkylene sulfosuccinate salts, the salts of the alkyl esters of polyoxyalkylene sulfosuccinic acid, the alkali metal salts of the polyoxyalkylene-modified dimethylpolysiloxane esters of sulfosuccinic acid, polyoxyalkylene alkylphenyl ether sulfate salts, long-chain alkanesulfonic acid salts, long-chain alkylsulfonates, polyoxyethylene alkylphenyl ether sulfate salts, polyoxyalkylene alkyl ether acetate salts, long-chain alkyl phosphate salts, polyoxyalkylene alkyl ether phosphate salts, acylglutamate salts, alpha-acylsulfonate salts, long-chain alkylsulfonate salts, alkylarylsulfonate salts, long-chain alpha-olefinsulfonate salts, alkylnaphthalenesulfonate salts, long-chain alkanesulfonic acid salts, long-chain alkyl or alkenyl sulfate salts, long-chain alkylamide sulfate salts, long-chain alkyl or alkenyl phosphate salts, alkylamide phosphate salts, alkyloylalkyltaurate salts, N-acylamino acid salts, sulfosuccinate salts, alkyl alkyl ether carboxylate salts, amide ether carboxylate salts, the salts of esters of alpha-sulfofatty acids, alanine derivatives, glycine derivatives, and arginine derivatives; salts can be exemplified by alkali-metal salts such as the sodium salt and potassium salt, alkanolamine salts such as the triethanolamine salt, and the ammonium salt, the sodium salt; alkyltrimethylammonium chloride, stearyltrimethylammonium chloride, lauryltrimethylammonium chloride, cetyltrimethylammonium chloride, beef tallow alkyltrimethylammonium chloride, behenyltrimethylammonium chloride, octyltrimethylammonium hydroxide, dodecyltrimethylammonium hydroxide, stearyltrimethylammonium bromide, behenyltrimethylammonium bromide, distearyldimethylammonium chloride, dicocoyldimethylammonium chloride, dioctyldimethylammonium chloride, di(POE)oleylmethylammonium (2EO) chloride, benzalkonium chloride, alkylbenzalkonium chloride, alkyldimethylbenzalkonium chloride, benzethonium chloride, stearyldimethylbenzylammonium chloride, lanolin-derived quaternary ammonium salts, diethylaminoethylamide of stearic acid, dimethylaminopropylamide of stearic acid, behenamidopropyldimethylhydroxypropylammonium chloride, stearoylcolaminoformylmethylpyridinium chloride, cetylpyridinium chloride, tall oil alkylbenzylhydroxyethylimidazolinium chloride, and benzylammonium salts; phospholipids, such as lecithin, phosphatidylethanolamine, phosphatidic acid, phosphatidylinositol, phosphatidylserine, phosphatidylcholine, phosphatidylglycerol, sphingomyelin, and cardiolipin, and the hydrogenates of the preceding. Particularly preferred are the hydrogenated natural lecithins as yielded by the hydrogenation of, for example, soy lecithin, egg yolk lecithin, corn lecithin, cottonseed oil lecithin, rapeseed lecithin; polyoxyalkylene ethers, polyoxyalkylene alkyl ethers, polyoxyalkylene fatty acid esters, polyoxyalkylene fatty acid diesters, polyoxyalkylene resin acid esters, polyoxyalkylene (hardened) castor oils, polyoxyalkylene alkylphenols, polyoxyalkylene alkylphenyl ethers, polyoxyalkylenephenyl phenyl ethers, polyoxyalkylene alkyl esters, polyoxyalkylene alkyl esters, sorbitan fatty acid esters, polyoxyalkylene sorbitan alkyl esters, polyoxyalkylene sorbitan fatty acid esters, polyoxyalkylene sorbitol fatty acid esters, polyoxyalkylene glycerol fatty acid esters, polyglycerol alkyl ethers, polyglycerol fatty acid esters, sucrose fatty acid esters, fatty acid alkanolamides, alkyl glucosides, polyoxyalkylene fatty acid bisphenyl ethers, polypropylene glycol, polyether-modified silicones (e.g., polyoxyalkylene-modified diorganopolysiloxanes, polyglycerol-modified silicones, glycerol-modified silicones, saccharide-modified silicones), perfluoropolyether-type surfactants, polyoxyethylene-polyoxypropylene block copolymers, alkyl polyoxyethylene-polyoxypropylene block copolymer ethers and a mixtures thereof.

### Humectant

The cosmetic composition of the invention further comprises the humectant. The humectant is such as sorbitol, mineral oil, vegetable oil, glycerol; betaine, guanidine, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, alkoxylated glucose, hyaluronic acid, lactamide monoethanolamine, acetamide monoethanolamine and derivatives, esters, salts and mixtures thereof; collagen, gelatin, aloe vera, hyaluronic acid or volatile water-soluble solvents, such as ethanol or propylene glycol.

### Emulsifier

The cosmetic composition of the invention may further comprise the emulsifier. The emulsifier is such as ethyl alcohol, isopropyl alcohol, ethyl carbonate, ethyl acetate, benzyl alcohol, benzyl benzoate, propylene glycol, 1,3-butylene glycol, oils (e.g., cottonseed, groundnut, corn, germ, olive, castor and sesame oils), glycerol, tetrahydrofuryl alcohol, polyethylene glycols and fatty acid esters of sorbitan, and mixtures thereof; carbomer, hydroxypropyl cellulose, sodium lauryl sulfate; glycerin fatty acid esters (monoglycerides, MG); mono- and di-glycerides (MG & DG) (e.g. Grindsted HV 40™ Poem J-2021™); distilled monoglycerides; citric acid esters of MG (CMG); diacetyl tartaric acid esters of mono- and di-glycerides (DATEMs) (e.g. Panodan AL 10™); polyglycerol esters of fatty acids (PGE); polyglycerol polyricinoleate (PGPR); sorbitan esters of fatty acids (e.g. Palsgaard 7463™), sucrose esters of fatty acids; calcium stearoyl lactylates; sodium stearoyl lactylates; lecithin (including enzyme digested lecithin); and caseinates (such as sodium caseinates including Alanate 191™); diacetyl tartaric acid esters of mono- and di-glycerides (DATEMs).

### Solubilizing Agent

The cosmetic composition of the invention further comprises the solubilizing agent. The solubilizing agent is such as citric acid, ethylenediamine-tetraacetate, sodium meta-phosphate, succinic acid, urea, cyclodextrin, polyvinylpyrrolidone, diethylammonium-ortho-benzoate, and micelle-forming solubilizers such as TWEEN® and spans, e.g., TWEEN 80®; polyoxyethylene sorbitan fatty acid ester, polyoxyethylene n-alkyl ethers, n-alkyl amine n-oxides, polyoxamers, organic solvents, such as acetone, phospholipids, cyclodextrin, triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200 to 600, glycofurol, transcutol, propylene glycol, and dimethyl isosorbide, miglyol, glycerin and glycerol.

### Solvent

The cosmetic composition of the invention further comprises the solvent. The solvent is such as methylene chloride; beta-cyclodextrin; dichloromethane; oily excipients or solvents are vegetable or animal oils, such as sunflower oil or cod liver oil; for aqueous or alcoholic solutions are water, ethanol, sugar solutions, or mixtures thereof; physiological saline solution such as glycerol; alcohols such as methanol, ethanol, propanol, isopropyl alcohol; sugar solutions such as glucose or mannitol solutions, or mixtures thereof; aromatic hydrocarbon solvents such as benzene, chlorobenzene, toluene and xylene; ether solvents such as diethyl ether, tert-butylmethyl ether, dimethoxyethane, tetrahydrofuran, dioxane and THF; aliphatic hydrocarbon solvents; ester solvents such as ethyl acetate; ketone solvents; chlorinated hydrocarbon solvents such as dichloromethane, chloroform and 1,2-dichloroethane; an organic solvent such as acetonitrile, 1,3-dimethyl-2-imidazolidinone, dimethylformamide, N-dimethylacetamide, N-methylpyrrolidine, dimethylsulfoxide, pyridine, nitromethane, mixtures thereof.

### Base Polymer

The cosmetic composition of the invention further comprises the base polymer. The base polymer is such as polysaccharide-based polymers, such as guar, xanthan and/or their derivatives; hydrophobic base polymers such as SIS (styrene/isoprene/styrene)-triblock copolymers, SBS (styrene/butadiene/styrene)-triblock copolymers, SBR (copolymers of styrene and butadiene), synthetic and/or natural polyisoprenes, polyamide, polyester, co-polyester, polyurethane and/or mixtures thereof are also possible as further matrices; water-soluble polymers, plant base polymers such as gum arabic, tragacanth gum, galacian, guar gum, carob gum, karaya gum, carragbeein, pectin, agar, quince seed (Marumero) algae colloid (seaweed extract), starch (rice, corn, potato, wheat), glycyrrhinic acid; microorganism base polymers such as xanthane gum, dextran, succinoglutan, pullulan; animal base polymers such as collagen, caseine, albumin, gelatin; starch base polymers such as carboxymethyl starch, methythydroxypropyl starch; cellulose base polymers such as methyl cellulose nitro cellulose, ethyl cellulose, methythydroxypropyl cellulose, hydroxyethyl cellulose, sodium cellulose sulfate, hydroxypropyl cellulose, sodium carboxymethyl cellulose (CMC), crystalline cellulose, cellulose powder; alginate base polymers such as sodium alginate, alginate propylene glycol esters; vinyl base polymers such as a polyvinyl alcohol, polyvinylmethyl ether, polyvinylpyrrolidone carboxyvinyl polymer (Carbopol), alkyl modified carboxyvinyl polymer, polyoxyethylene base polymers such as polyethylene glycol 2000, 4000, 6000; acryl base polymers such as polyacrylates or salt thereof, polyoxyethylene polyoxypropylene copolymer brae polymer, sodium polyacrylate, polyethylene acrylate, polyacryl amide, polyethylene imine, cationic polymer, etc. may be mentioned.

### Diluent

The cosmetic composition of the invention further comprises the diluent. The diluent is such as water, saline, finger's solutions, dextrose solution; calcium carbonate, sodium carbonate, lactose, calcium phosphate or sodium phosphate; granulating and disintegrating agents, e.g., corn starch or alginic acid; binding agents, for example starch gelatin, acacia, microcrystalline cellulose or polyvinyl pyrrolidone; dicalcium phosphate, calcium sulfate, lactose, sorbitol, sucrose, inositol, cellulose, kaolin, mannitol, sodium chloride, dry starch, and powdered sugar.

### Antioxidant

The cosmetic composition of the invention further comprises the antioxidant. The antioxidant is such as sodium bisulfite, butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

### Fragrance

The cosmetic composition of the invention further comprises the fragrance. The natural fragrance is such as buttery, banana, almond, bitter almond, cherry, cinnamon, fruity, grape, orange, pear, pineapple, sugar, cotton candy, vanilla, wintergreen, minty, apple, rosemary, lavender, ginseng, musk, green tea, violet, lily, lemon, rose, jasmine, blueberry, peach, coconut, orange, mandarin, jam, apricot, fennel, honey, plum, raspberry, alcohol, etc. The artificial fragrance is such as benzaldehyde, p-tolyl aldehyde, decyl aldehyde, cinnamic aldehyde, ionone, gamma undecalactone, anethole, malonates, phenylacetate acid ester, ethyl acid ester, cyclohexyl cinnamate, ethyl acetosuccinate, citronella, geraniol, linalool, etc.

### Preservative

The cosmetic composition of the invention further comprises the preservative. The preservative is such as methyl hydroxy benzoate, propyl hydroxy benzoate, chorocresol, benzoic acid and phenyl mercuric nitrate.

### Method of Preparation

The cosmetic composition of the present invention is prepared using the known methods or by adopting specific conditions suitable for the ingredients employed. Examples of the cosmetic compositions and the amount of the ingredients are listed in Table I below, but not

TABLE 1

| Ingredients | Ratio (% w/w) |
|---|---|
| Kava Extract 80% | 0-5 |
| Kava Extract 60% | 0-10 |
| Dipalmitoyl Hydroxyproline | 0-2 |
| Sodium Hyaluronate | 0-0.2 |
| Glycerin/Butylene Glycol/Water/ | 0-5 |
| Carbomer/Polysorbate-20/Palmitoyl | |
| Pentapeptide-3 | |
| Sucrose Fatty Acid Ester | 0-2 |
| Enhancers (selected from | 0-8 |
| Diethylene Glycol Monoethyl Ether, | |
| Caprylocaproyl Polyoxylglycerides, | |
| Propylene Glycol Monolaurate, | |
| Polyglyceryl Dioleate, Diisopropyl | |
| Adipate, or Sucrose Ester D1811) | |
| Medium-Chain Triglycerides | 2-8 |
| Cetearyl Alcohol/Cetearyl Glucoside | 1-3 |
| Glyceryl Stearate/PEG-100 Stearate | 0.5-2 |
| Glycol Palmitate | 0.5-2 |
| Cetearyl Alcohol | 0.5-2 |
| Squalane | 2-8 |
| Dimethicone | 1-3 |
| Tocopheryl Acetate | 0.2-0.8 |
| Propylparaben | 0.05-0.2 |
| Polyacrylate13/Polyisobutene/ | 0.5-1.1 |
| Polysorbate-20 | |
| Methylparaben | 0.1-0.3 |
| Allantoin | 0.1-0.3 |
| Glycerin | 2-8 |
| 1,3-Butylene Glycol | 1.5-4.5 |
| Disodium EDTA | 0.05-0.2 |
| Xanthan Gum | 0.1-0.3 |
| Propylene Glycol/Diazolidinyl | 0.2-0.8 |
| Urea/Iodopropynyl Butylcarbamate | |
| Portulaca Oleracea Extract | 1-3 |
| Polyethylene (21) Stearyl Ether | 0-0.8 |
| Isopropyl Myristate | 0-3 |
| Tween 80 | 0-3 |
| Flavour | 0-0.3 |
| CoQ 10 | 0-0.3 |
| Water | 55-70 |
| Total (% w/w) | 100 |

### Method of Reducing the Appearance of Wrinkles

To reduce the appearance of wrinkles and lines in a person's skin, the cosmetic composition is applied topically on the skin of a subject in need thereof. The active agent wherein Kavalactones is delivered to and absorbed by the skin and absorbed systemically. The total amount of the Kavalactones delivered is at least a fraction of 1% to 80% or greater of the composition.

### EXAMPLES

The following examples are merely exemplary in nature and is not intended to limit application and uses. The following examples further illustrate the present invention without, however, limiting the scope of the invention thereto. Various changes and modifications can be made by those skilled in the art on the basis of the description of the invention, and such changes and modifications are also included in the present invention.

### Example 1

### Kava Anti-Wrinkle Cream Formulation

Kava anti-wrinkle cream were prepared according to the components and amounts shown in Table 2.

TABLE 2

| | Example (% by weight) | | | | | | |
|---|---|---|---|---|---|---|---|
| Ingredients | A | B | C | D | E | F | G |
| Kava Extract 80% | 3 | 3 | 3 | 0 | 0 | 0 | 3 |
| Kava Extract 60% | 0 | 0 | 0 | 2 | 4 | 8 | 0 |
| Dipalmitoyl Hydroxyproline | 1 | 0 | 0 | 0 | 0 | 0 | 0 |
| Sodium Hyaluronate | 0.1 | 0 | 0 | 0 | 0 | 0 | 0 |
| Glycerin/Butylene | 3 | 0 | 0 | 0 | 0 | 0 | 0 |
| Glycol/Water/Carbomer/Polysorbate- | | | | | | | |
| 20/Palmitoyl Pentapeptide-3 | | | | | | | |
| Sucrose Fatty Acid Ester | 0 | 1.5 | 0 | 0 | 0 | 0 | 0 |
| Enhancers (selected from Diethylene | 0 | 0 | 0 | 5 | 5 | 5 | 0 |
| Glycol Monoethyl Ether, | | | | | | | |
| Caprylocaproyl Polyoxylglycerides, | | | | | | | |
| Propylene Glycol Monolaurate, | | | | | | | |
| Polyglyceryl Dioleate, Diisopropyl | | | | | | | |
| Adipate, or Sucrose Ester D1811) | | | | | | | |
| Medium-Chain Triglycerides | 5 | 5 | 5 | 5 | 5 | 5 | 0 |
| Cetearyl Alcohol/Cetearyl Glucoside | 2 | 2 | 2 | 2 | 2 | 2 | 2 |
| Glyceryl Stearate/PEG-100 Stearate | 1 | 1 | 1 | 1 | 1 | 1 | 1 |
| Glycol Palmitate | 1 | 1 | 1 | 1 | 1 | 1 | 1 |
| Cetearyl Alcohol | 1 | 1 | 1 | 1 | 1 | 1 | 1 |
| Squalane | 5 | 5 | 5 | 5 | 5 | 5 | 5 |
| Dimethicone | 2 | 2 | 2 | 2 | 2 | 2 | 2 |
| Tocopheryl Acetate | 0.5 | 0.5 | 0.5 | 0.5 | 0.5 | 0.5 | 0.5 |
| Propylparaben | 0.1 | 0.1 | 0.1 | 0.1 | 0.1 | 0.1 | 0.1 |
| Polyacrylate13/Polyisobutene/ | 0.8 | 0.8 | 0.8 | 0.8 | 0.8 | 0.8 | 0.8 |
| Polysorbate-20 | | | | | | | |
| Methylparaben | 0.2 | 0.2 | 0.2 | 0.2 | 0.2 | 0.2 | 0.2 |
| Allantoin | 0.2 | 0.2 | 0.2 | 0.2 | 0.2 | 0.2 | 0.2 |
| Glycerin | 5 | 5 | 5 | 5 | 5 | 5 | 5 |
| 1,3-Butylene Glycol | 3 | 3 | 3 | 3 | 3 | 3 | 3 |
| Disodium EDTA | 0.1 | 0.1 | 0.1 | 0.1 | 0.1 | 0.1 | 0.1 |
| Xanthan Gum | 0.2 | 0.2 | 0.2 | 0.2 | 0.2 | 0.2 | 0.2 |
| Propylene Glycol/Diazolidinyl | 0.5 | 0.5 | 0.5 | 0.5 | 0.5 | 0.5 | 0.5 |
| Urea/Iodopropynyl Butylcarbamate | | | | | | | |
| Portulaca Oleracea Extract | 2 | 2 | 2 | 2 | 2 | 2 | 2 |
| Polyethylene (21) Stearyl Ether | 0 | 0.5 | 0 | 0 | 0 | 0 | 0 |
| Isopropyl Myristate | 0 | 1 | 0 | 0 | 0 | 0 | 0 |
| Tween 80 | 0 | 1 | 0 | 0 | 0 | 0 | 0 |
| Flavour | 0 | 0.1 | 0 | 0 | 0 | 0 | 0 |
| CoQ 10 | 0 | 0.1 | 0 | 0 | 0 | 0 | 0 |
| Water | 63.3 | 63.2 | 67.4 | 63.4 | 61.4 | 57.4 | 72.4 |
| Total (% w/w) | 100 | 100 | 100 | 100 | 100 | 100 | 100 |

### Example 2

### Immediate Anti-Wrinkle Effect of Kava Cream

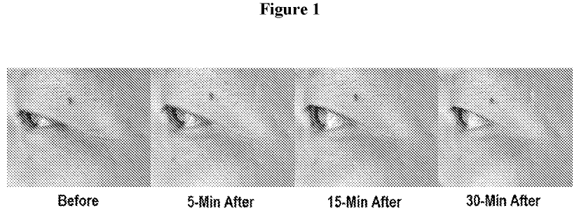

The formulation C was applied around the eyes of the subject. After application of the formulation C, the crow s feet were diminished in 5 minutes and the effect was more prominent 30 minutes afterwards. Images in FIG. 1 show immediate anti-wrinkle effect of the invention.

### Example 3

### Long-Term Anti-Wrinkle Effect of Kava Cream

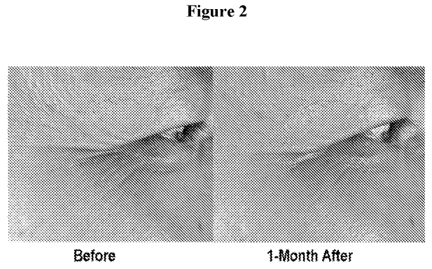

The formulation C was applied twice daily around the eyes of the subject for one month. The crow s feet and fine lines around the eyes were reduced distinctly after one-month application. Images in FIG. 2 show long-term anti-wrinkle effect of the invention.

### Example 4

### Effect Of The Penetration Enhancer in Long-Term Anti-Wrinkle Effect of Kava Cream

TABLE 3
Standard Wrinkle Grades (Change from baseline)

| | Formulation G | Formulation C |
|---|---|---|
| | (without enhancer) | (enhancer included) |
| Application Site | Left Eye | Right Eye |
| Before Use | 3.0 | 3.0 |
| Day 1 | 3.0 | 2.8 |
| Day 2 | 2.8 | 2.6 |
| Day 3 | 2.8 | 2.4 |
| Day 4 | 2.8 | 2.2 |
| Day 5 | 2.6 | 2.0 |
| Day 6 | 2.6 | 2.0 |

The formulation containing enhancer (Formulation C) and no enhancer (Formulation G) as shown in Table 2 were applied twice daily around each eyes of the subject for 6 days. The standard wrinkle grade dropped by 33% from 3.0 to 2.0 for Kava cream included enhancer compared to 13.3% from 3.0 to 2.6 for Kava cream without enhancer. It shown that the penetration enhancer expedited the Kava cream's anti-wrinkle effect.

### Example 5

### Efficacy of Kava Cream on the Wrinkles

A randomized, single-blinded controlled, pilot efficacy study determined the positive outcome of Kava cream on wrinkles around the eyes. Thirty female volunteers whose wrinkle score (Bissett D L et al. Dermatol Surg 2005; 31: 860-865) was 2 or above were enrolled. Formulation C was applied twice daily for 3 months. The efficacies were evaluated at 0 week, 2 weeks, 4 weeks, 6 weeks, 8 weeks, and 12 weeks.

The formulation C was shown to exert a fast onset of anti-wrinkle effect. After 15min of the first application, wrinkle score of subjects decreased by 1.63 and wrinkle was diminished significantly in 63.3% (assessment by investigators) and 50.0% (self-assessment by subjects) of subjects compared with 0.10% and 0% for placebo group. After 12 weeks, the wrinkle score of subjects decreased by 1.90, while that for placebo group was only 1.21. Around 90% of subjects was deemed significant reduction of wrinkle, while that for placebo group was only around 30%.

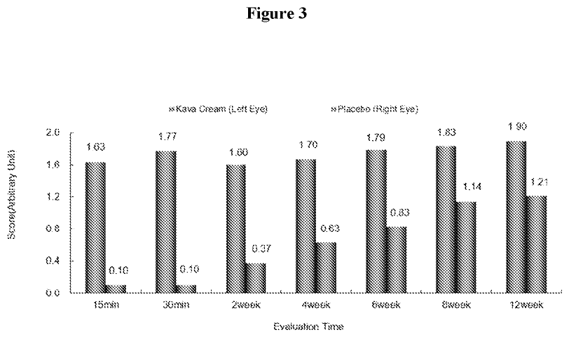

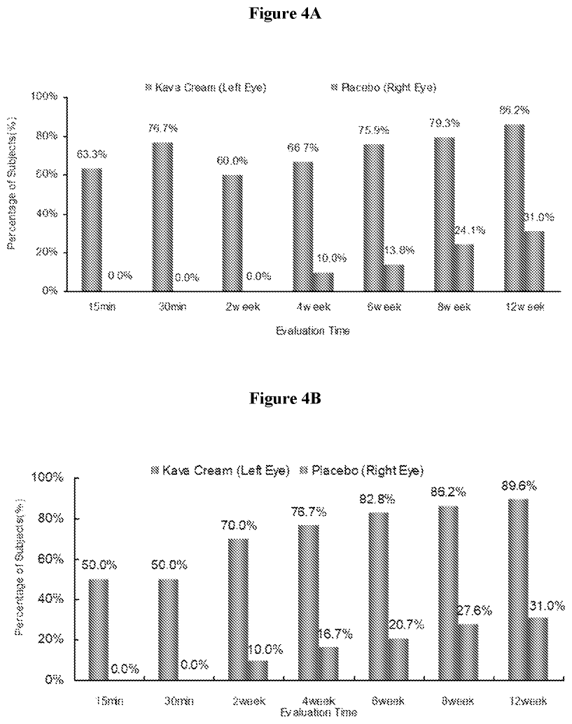

The change of wrinkle scores is shown in FIG. 3. FIGS. 4A and 4B show the percentage of subjects whose wrinkle score was dropped by 2 evaluated by investigators (FIG. 4A) or by the subjects (FIG. 4B). The results have revealed the short-term and long-term effect of Kava cream on diminishing wrinkle around the eyes.

### Example 6

### Efficacy of Kava Cream on the Water Content of Skin Around the Eyes

A randomized, single-blinded controlled, pilot efficacy study determined the positive outcome of Kava cream on the water content of skin around the eyes. Thirty female volunteers whose wrinkle score (Bissett D L et al. Dermatol Surg 2005; 31: 860-865) was 2 or above were enrolled. Formulation C was applied twice daily for 3 months. The efficacies were evaluated at 0 week, 2 weeks, 4 weeks, 6 weeks, 8 weeks, and 12 weeks.

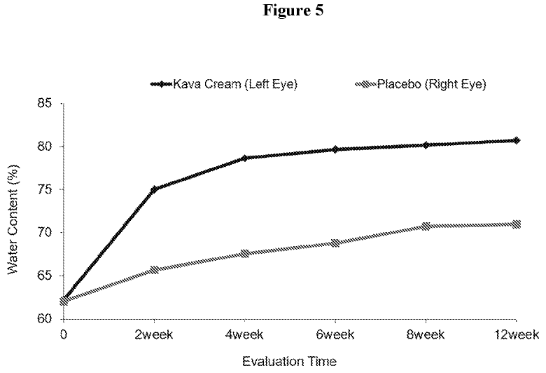

After application of formulation C, the water content was increased by 12.83% from 62.20% to 75.03% in 2 weeks, which is significantly higher than the 3.64% for placebo group. The improvement continued to grow with time until the 12-week; the water content of Kava cream group reached 80.69%, increased by 18.49%, while that of placebo group only reached 70.97, increased by 8.77%. FIG. 5 shows that Kava cream promotes water content of skin with time.

### Example 7

### Efficacy of Kava Cream on the Elasticity of Skin Around the Eyes

A randomized, single-blinded controlled, pilot efficacy study determined the positive outcome of Kava cream on the elasticity of skin around the eyes. Thirty female volunteers whose wrinkle score (Bissett DL et al. Dermatol Surg 2005; 31: 860-865) was 2 or above were enrolled. Formulation C was applied twice daily for 3 months. The efficacies were evaluated at 0 week, 2 weeks, 4 weeks, 6 weeks, 8 weeks, and 12 weeks.

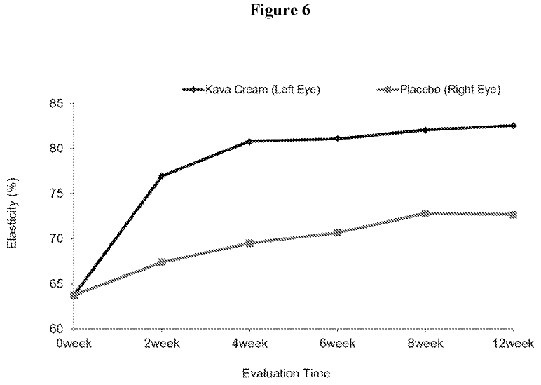

After application of formulation C, the elasticity was increased by 13.2% from 63.77% to 76.97% in 2 weeks, which is significantly higher than the 3.67% for placebo group. The improvement mounted with time until the 12-week; the elasticity of Kava cream group reached 82.55%, increased by 18.78%, while that of placebo group only reached 72.69%, increased by 8.92%. FIG. 6 demonstrates Kava cream improves elasticity of skin with time. In addition, 86.2% of subjects had wrinkles around the eyes ameliorated.

The above clinical studies showed that Kava cream is a safe topical product that have a strong anti-wrinkle effect with fast onset of action in short-term and long-term.

1. A cosmetic composition comprising a Kava extract to reduce the appearance of wrinkles and lines in a person's skin.

2. The cosmetic composition of claim 1, wherein said the Kava extract is extracted from Piper Methysticum root, leaf, stem, or their combinations thereof by using polar solvents, non-polar solvents, or supercritical fluid such as carbon dioxide.

3. The cosmetic composition of claim 1, wherein the active substance in the Kava extract is Kavalactones.

4. The cosmetic composition of claim 4, wherein the amount of Kavalactones is about 1% to about 90% of the total weight of the extract.

5. The cosmetic composition of claim 1, further comprising other anti-wrinkle agents selected from the group consisting of dipalmitoyl hydroxyproline, sodium hyaluronate, palmitoyl pentapeptide-3, acetyl hexapeptide-3, acetyl hexapeptide-8, and CoQ 10.

6. The cosmetic composition of claim 1, further comprising one or more penetration enhancers.

7. The cosmetic composition of claim 6, wherein the penetration enhancers have a combined HLB value of about 1 to about 16.

8. The cosmetic composition of claim 1, further comprising a surfactant, a humectant, an emulsifier, a solubilizing agent, a solvent, a base polymer, a diluent, an antioxidant, a fragrance, a secondary penetration enhancer or a preservative.

9. The cosmetic composition of claim 6, wherein said penetration enhancers are selected from the group comprising PEG-8 beeswax, PEG-75 stearate, pegoxol-7 stearate, propylene glycol monocaprylate, propylene glycol monolaurate, propylene glycol monostearate, propylene glycol dioleate, 2-hydroxypropyl stearate, 2-hydroxypropyl laurate, propylene glycol oleate, propylene glycol distearate, propylene glycol dicaprylate, propylene glycol dilaurate, polypropylene glycol (17) dioleate, propyleneglycol monolaurate, propylene glycol monomyristate, dipropylene glycol dipelargonate, polypropylene glycol monobutyl ether oleate, propylene glycol dipelargonate, propylene glycol didecanoate, dipropylene glycol dipelargonate, propylene glycol bis(9,10-epoxystearate), propylene glycol monoisostearate, propylene glycol diundecanoate, glycol monoethyl ether, diethylene glycol monobutyl ether, oleoyl macrogolglycerides, lauroyl macrogolycerides, stearoyl macrogolgylycerides, caprylocaproyl macrogolglycerides, medium-chain triglycerides, polyglyceryl-3 diisostearate, polyglyceryl oleate, ethylene glycol paimitostearate, dissopropyl adipate, di-nbutyl adipate, dimethyl adipate, dimethyl isosorbide, or diethylene glycol monoethyl ether.

10. The cosmetic composition of claim 6, wherein said one or more penetration enhancers is about 0.1% to about 20%, about 0.1% to about 15%, or about 1% to about 10% of the total weight of the composition.

11. The cosmetic composition of claim 8, wherein the secondary penetration enhancer selected from the group consisting of glycerol and terpenes.

12. The cosmetic composition of claim 1, wherein the cosmetic composition is in the form of a product selected from the group comprising O/W and W/O emulsions, lotions, ointments, paste, solutions, lacquers, creams or gels.

13. A method of reducing the appearance of wrinkles and lines in a person's skin, the method comprising;
topically applying the cosmetic composition of claim 1 to skin in need thereof, wherein at least a fraction of 1% to 80% or greater of the Kavalactones is delivered to and absorbed by the skin and absorbed systemically.
